(19) United States
(12) Patent Application Publication
(10) Pub. No.: US 2005/0260286 A1
(43) Pub. Date: Nov. 24, 2005

(54) Intensive anti-cellulite formula

(76) Inventor: Giovina DiMateeo-Leggio, Aventura, FL (US)

Correspondence Address: GIOVINA INTERNATIONAL INC. OF AMERICA, SUITE #807, 19495 BISCAYNE BLVD., AVENTURA, FL, 33180, US

(21) Appl. No.: 10/628,346

(22) Filed: May 21, 2004

(51) Int. Cl. A61K 35/78

(52) U.S. Cl. 424/725

(57) ABSTRACT

The supplements are a combination of natural herbs, vitamins and minerals that help helps to significantly reduce the appearance of cellulite while detoxifying the body. They are packaged in bottles of 180 tablets. The supplements are in the form of tablets of which six tablets daily are taken orally.

ANTI-CELLULITE INTENSIVE THERAPY
SUPPLEMENT FACTS

| Supplement Size 6 tablets | Serving per | | % Daily |
|---|---|---|---|
| Amount per serving | container 30 | | Value |
| Vitamin C (Ascorbic Acid) | 500 | mg | 833% |
| Vitamin B6 (Pyridoxine Hydrochloride) | 15 | mg | 750% |
| Vitamin E (Succinate) | 150 | IU | 500% |
| Chromium (Amino Acid Chelate) | 100 | mcg | 83% |
| Forslean (Coleus forskohlii, standaidized to | 100 | mg | |
| contain 10% forskohlin) | | | |
| Ginkgo Biloba (standardized to contain 24% | 20 | mg | |
| ginkgo flavone glycosides, 6% terpene lactones) | | | |
| Bladderwrack (Fucus vesiculosus) | 50 | mg | |
| Gotu Kola Extract (Centella asiatica, standardized | 100 | mg | |
| to contain 70% triterpenes) | | | |
| Cinnamon (Cinnamomum verum) | 50 | mg | |
| Grape Seed (Vitis vinifera, standardized to | 150 | mg | |
| contain 86% oligomeric procyanidolic complexes) | | | |
| Kelp (Laminaria hyperborea) | 100 | mg | |
| Evening Primrose (Oenothera biennis) | 200 | mg | |
| Sage Leaf (Salvia officianales) | 100 | mg | |
| Uva Ursi (Arctostaphylos uva-ursi) | 200 | mg | |
| Lecithin | 300 | mg | |
| Senna Leaf (Cassia species) | 50 | mg | |
| Cascara Sagrada (Rhamnus purshiana) | 50 | mg | |
| 5-Hydroxy Tryptophan | 1000 | mg | |
| Dandilion (Taraxacum offcinales) | 100 | mg | |
| Curcumin | 500 | mg | |
| Bioperine (Piper nigrum) | 5 | mg | |
| L-Camitine (Tartrate) | 100 | mg | |
| Conjugated Linolenic Acid | 1000 | mg | |

% Daily Value not to established
Directions: Take 6 tablets daily. Preferably with meals.
180 TABS PER BOTTLE
Other Ingredients: Calcium Carbonate, Dibasic Calcium Phosphate, Microcrystalline Cellulose, Croscarmellose Sodium, Modified Cellulose, Stearic Acid, Magnesium Stearate, Silicon Dioxide, Hydroxypropyl Methylcellulose, Water

ANTI-CELLULITE INTENSIVE THERALY - TOPICAL
ESSENCIAL OIL
INGREDIENTS AT 24% CONCENTRATION
Black Pepper, Juniper, Patchouli, Lavender, Jojoba Carrier Oil Base
Natural Fiber Brush

1. I claim that our Anit-Cellulite Formula helps to significantly reduce the appearance of cellulite while detoxifying the body. So effective that results can be observed within two weeks no matter how severe and resistant the cellulite. This is a complete, exclusive 4-stage program consisting of a nutritional food plan by stages; a totally individualized supplement made of 100% natural herb of lipotropic, antioxidant, detoxifying an lymphatic components; fitness plans; dry body brush and routine; a customized preparation of topical essential oil blends.
